SEQUENCE LISTING (21 sequences)

SEQ ID NO: 1 - DNA, 576 bp, synthetic construct
  source 1..576: mol_type = other DNA; note = PCV2 Cap gene
aatggcatcttcaacacccgcctctcccgcaccttcggatatactatcaagcgaaccacagtcaaaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaatcagctgtggctgagactacaaactgctggaaatgtggaccacgtaggcctcggcactgcgttcgaaaacagtatatacgaccaggaatacaatatccgtgtaaccatgtatgtacaattcagagaatttaatcttaaagaccccccacttaaccct

SEQ ID NO: 2 - protein, 192 aa, synthetic construct
  source 1..192: note = dCap protein
NGIFNTRLSRTFGYTIKRTTVKTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 3 - protein, 579 aa, synthetic construct
  source 1..579: note = amino acid sequences of the gE gene
MRPFLLRAAQLLALLALALSTEAPSLSAETTPGPVTEVPSPSAEVWDDLSTEADDDDLNGDLDGDDRRAGFGSALASLREAPPAHLVNVSEGANFTLDARGDGAVLAGIWTFLPVRGCDAVSVTTVCFETACHPDLVLGRACVPEAPEMGIGDYLPPEVPRLRREPPIVTPERWSPHLSVLRATPNDTGLYTLHDASGPRAVFFVAVGDRPPAPADPVGPARHEPRFHALGFHSQLFSPGDTFDLMPRVVSDMGDSRENFTATLDWYYARAPPRCLLYYVYEPCIYHPRAPECLRPVDPACSFTSPARARLVARRAYASCSPLLGDRWLTACPFDAFGEEVHTNATADESGLYVLVMTHNGHVATWDYTLVATAAEYVTVIKELTAPARAPGTPWGPGGGDDAIYVDGVTTPAPPARPWNPYGRTTPGRLFVLALGSFVMTCVVGGAIWLCVLCSRRRAASRPFRVPTRARTHMLSPVYTSLPTHEDYYDGDDDDDEEAGVIRRRPASPSGDSGYEGPYASLDPEDEFSSDEDDGLYVRPEEAPRSGFDVWFRDPEKPEVTNGPNYGVTANRLLMSRPA

SEQ ID NO: 4 - DNA, 5549 bp, synthetic construct
  source 1..5549: mol_type = other DNA; note = Transfer vector
cacgtcgccggcagcgccgtcctcgtgcccggcgacgcgcccaacctgacgatagacgggacgctgctgtttctggaggggccctcgccgagcaactacagcgggcgcgtggagctgctgcgcctcgaccccaagcgcgcctgctacacgcgcgagtacgccgccgagtacgacctctgcccccgcgtgcaccacgaagccttccgcggctgcctgcgcaagcgcgagccgctcgcccggcgcgcgtccgccgcggtggaggcgcgccggctgctgttcgtctcgcgcccggcctcgggggacgcggggtcgtacgtgctgcgggtccgcgtgaacgggaccacggacctctttgtgctgacggccctggtgccgccgagggggcgccccgccaccacgtcgccgcccgcggacgagtgccggcccgtcgtcggatcgtggcacgacagcctgcgcgtcgtggaccccgccgaggacgccgtgttcaccacccagcccccgcccgagcccgagccgccgacgacccccgcgcccccccgggggaccggcgccacccccgagccccgatcggacgaggaggaggagggtgacgcggagacgacgacgccgacgctgaccccggcgcccgggaccctggacgcgaacggcacgatggtgctgaacgccagcgtcgtgtcgcgcgtcctgctcgccgccgccaacgccacggcgggcgcccggagccccgggaagatagccatggtgctggggcccacgatcgtcgtcctcctgatcttcctgggcgggatcgcctgcgtggcccggcgctgcgcgcggaatcgcatctaccggccgcgacccgggcgcggatcggcggtccatgcggcgcccccgcggcgcccgccccccaaccccgtcgccggggcgcccgtcccccagcccaagatgacgttggccgagctgcgccagaagctcgccaccatcgcagaagaacaataaaaaggtggtgtttgcataattttgtgggtggcgttttatctccgtccgcgccgttttaaacctgggcacccccgcgagtctcgcacacaccggggttgagacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgccaccatgcggccctttctgctgcgcgccgcgcagctcctggcgctgctggccctggcgctctccaccgaggccccgagcctctccgccgagacgaccccgggccccgtcaccgaggtcccgagtccctcggccaatggcatcttcaacacccgcctctcccgcaccttcggatatactatcaagcgaaccacagtcaaaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaatcagctgtggctgagactacaaactgctggaaatgtggaccacgtaggcctcggcactgcgttcgaaaacagtatatacgaccaggaatacaatatccgtgtaaccatgtatgtacaattcagagaatttaatcttaaagaccccccacttaaccctggcggcggcgacgacgcgatctacgtggacggcgtcacgacgccggcgccgcccgcgcgcccgtggaacccgtacggccggacgacgcccgggcggctgtttgtgctggcgctgggctccttcgtgatgacgtgcgtcgtcgggggggccatctggctctgcgtgctgtgctcccggcgccgggcggcctcgcggccgttccgggtgccgacgcgggcgcggacgcacatgctctctccggtgtacaccagcctgcccacgcacgaggactactacgacggcgacgacgacgacgacgaggaggcgggcgtcatccgccggcggcccgcctcccccagcggagacagcggctacgaggggccgtacgcgagcctggaccccgaggacgagttcagcagcgacgaggacgacgggctgtacgtgcgccccgaggaggcgccccgctccggcttcgacgtctggttccgcgatccggagaaaccggaagtgacgaatggacccaactatggcgtgaccgccaaccgcctgttgatgtcccgccccgcttaaataacttcgtataatgtatgctatacgaagttatgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagataacttcgtataatgtatgctatacgaagttatataccgggagaaccggtccgcccgcattccgacatgcccggcgccgcctccgtcgacatggacacgtttgaccccagcgcccccgtcccgacgagcgtctcgaacccggccgccgacgtcctgctggcccccaagggaccccgctccccgctgcgcccccaggacgactcggactgctactacagcgagagcgacaacgagacgcccagcgagttcctgcgccgcgtgggacgccggcaggcggcgcgtcggagacgccgccgctgcctgatgggcgtcgcgatcagcgccgccgcgctggtcatctgctcgctgtccgcgctactcgggggcatcgtcgccaggcacgtgtagcgagcgagcgagcgaacgggagcgggggcccgcccccatccgccgcgcccaggagaggggggagagagcggggggttgggcgcgccacgtggtgtgggcacggactcggacttgtcacaataaatgggccccggcgtgtccgggcgcacacagcagccttcctctcctccgcgtctctgttccgcccgtctctcgccggactcttcttctccaccgcctccaccgtcgcagttgtcgcgagcgcgttcgcaccatgggggtgacggccatcaccgtggtcacgctgatggacggggccgggcgcatccccgccttcgtgggcgaggcgcacccggacctgtggaaggtgctcaccgagtggtgctacgcgtcgatggtgcagcagcggcgcgccgccgacgagaactcgccgcggcagcacgtggtgctgcgctcctcggagatctcccccggctcgctggccctgctgccgcgcgccgtgcgccccgtcgtgcggacgcggtccgaccccacggcgccgttctacatcaccaccgagacgcacgagctgacgcggcgccccccggcggacggctcgaagcccggggagcccctcaggatcagcccacccccgcggctggacacggagtggtcgtccgtcctgaacgggatccagtacctgaactcgggggcccggggcacggcccccgtccacctgtggatcctgggcgccgccgacctctgcgaccaggtgctcctggccgcctcccgcagcaccgccgccggagcctcccacgcccagacgggcgcgcgcctgacccggcgccggcccgggctgacggacgccgacgccctggacgtgatcgtcgccgggatccaggcgacccgcgccatgttcgcgcgggtccacaac

SEQ ID NO: 5 - DNA, 42 bp, synthetic construct
  source 1..42: mol_type = other DNA; note = Primer LgE-F-pUC18 plus
gaattcgagctcggtacccacgtcgccggcagcgccgtcctc

SEQ ID NO: 6 - DNA, 40 bp, synthetic construct
  source 1..40: mol_type = other DNA; note = Primer LgE-R-CMV plus
ttgattactattaataactaggtctcaaccccggtgtgtg

SEQ ID NO: 7 - DNA, 44 bp, synthetic construct
  source 1..44: mol_type = other DNA; note = Primer CMV-F-LgE plus
cacacaccggggttgagacctagttattaatagtaatcaattac

SEQ ID NO: 8 - DNA, 49 bp, synthetic construct
  source 1..49: mol_type = other DNA; note = Primer CMV-R-gESP plus
cgcagcagaaagggccgcatggtggcgatctgacggttcactaaaccag

SEQ ID NO: 9 - DNA, 40 bp, synthetic construct
  source 1..40: mol_type = other DNA; note = Primer gESP-F-CMV plus
gtgaaccgtcagatcgccaccatgcggccctttctgctgc

SEQ ID NO: 10 - DNA, 47 bp, synthetic construct
  source 1..47: mol_type = other DNA; note = Primer gESP-R-2dCap plus
cgggtgttgaagatgccattggccgagggactcgggacctcggtgac

SEQ ID NO: 11 - DNA, 46 bp, synthetic construct
  source 1..46: mol_type = other DNA; note = Primer 2dCap-F-gESP plus
aggtcccgagtccctcggccaatggcatcttcaacacccgcctctc

SEQ ID NO: 12 - DNA, 44 bp, synthetic construct
  source 1..44: mol_type = other DNA; note = Primer 2dCap-R-gEKB plus
atcgcgtcgtcgccgccgccagggttaagtggggggtctttaag

SEQ ID NO: 13 - DNA, 44 bp, synthetic construct
  source 1..44: mol_type = other DNA; note = Primer gEKB-F-2dCap plus
aagaccccccacttaaccctggcggcggcgacgacgcgatctac

SEQ ID NO: 14 - DNA, 43 bp, synthetic construct
  source 1..43: mol_type = other DNA; note = Primer gEKB-R-pUC18 plus
gacggccagtgccaagcttttaagcggggcgggacatcaacag

SEQ ID NO: 15 - DNA, 51 bp, synthetic construct
  source 1..51: mol_type = other DNA; note = Primer gEKB-R-loxP plus
ataacttcgtatagcatacattatacgaagttatttaagcggggcgggaca

SEQ ID NO: 16 - DNA, 45 bp, synthetic construct
  source 1..45: mol_type = other DNA; note = Primer EGFP-F
tgtatgctatacgaagttatgcccctctccctcccccccccctaa

SEQ ID NO: 17 - DNA, 49 bp, synthetic construct
  source 1..49: mol_type = other DNA; note = Primer EGFP-R-loxP plus
ataacttcgtatagcatacattatacgaagttatctacttgtacagctc

SEQ ID NO: 18 - DNA, 38 bp, synthetic construct
  source 1..38: mol_type = other DNA; note = Primer RgE-F-loxP plus
tgtatgctatacgaagttatataccgggagaaccggtg

SEQ ID NO: 19 - DNA, 44 bp, synthetic construct
  source 1..44: mol_type = other DNA; note = Primer RgE-R-pUC18 plus
gacggccagtgccaagcttgttgtggacccgcgcgaacatggcg

SEQ ID NO: 20 - DNA, 22 bp, synthetic construct
  source 1..22: mol_type = other DNA; note = Primer gE-US7-9 F
atcttcctgggcgggatcgcct

SEQ ID NO: 21 - DNA, 25 bp, synthetic construct
  source 1..25: mol_type = other DNA; note = Primer gE-US7-9 R
agatgaccagcgcggcggcgctgat

(19) United States
(12) Patent Application Publication
(10) Pub. No.: US 2024/0398937 A1
(43) Pub. Date: Dec. 5, 2024

(54) PSEUDORABIES VIRUS (PRV) EXPRESSING PORCINE CIRCOVIRUS 2 (PCV2) CAPSID PROTEIN ON ENVELOPE AND USE THEREOF

(71) Applicant: ZHEJIANG UNIVERSITY, Hangzhou City (CN)

(72) Inventors: Jiyong ZHOU, Hangzhou City (CN); Yulan JIN, Hangzhou City (CN); Weiren DONG, Hangzhou City (CN); Yan YAN, Hangzhou City (CN); Jinyan GU, Hangzhou City (CN)

(73) Assignee: ZHEJIANG UNIVERSITY, Hangzhou City (CN)

(21) Appl. No.: 18/602,029

(22) Filed: Mar. 12, 2024

(30) Foreign Application Priority Data
May 31, 2023 (CN) 202310632792.3

(51) Int. Cl. A61K 39/245; A61K 39/00; C12N 7/00; C12N 9/12

(52) U.S. Cl. CPC A61K 39/245; C12N 7/00; C12N 9/1241; A61K 2039/70; C12N 2710/00034; C12N 2710/16721; C12N 2710/16722; C12N 2710/16734; C12N 2710/16743

(57) ABSTRACT

The present disclosure provides a pseudorabies virus (PRV) expressing a porcine circovirus 2 (PCV2) capsid protein in its envelope, and use thereof. In the present disclosure, an attenuated PRV vaccine strain is used as a vector to express an exogenous immunogen. The exogenous immunogen only replaces an extracellular domain of a non-essential envelope protein of the PRV, while retaining a transmembrane domain and an intracellular domain of an original envelope protein, thereby allowing the expression of one or more exogenous immunogens on the viral envelope without changing genes of other PRV autoimmunogens. During the recombinant PRV particle being recognized by the body's immune system, the host's immune system recognizes all immunogens (exogenous immunogens and PRV autoimmunogens) on the recombinant PRV particle and then initiates an immune response, thus exerting a protective effect of a bivalent vaccine or a polyvalent vaccine based on the recombinant PRV particle.

### CROSS REFERENCE TO RELATED APPLICATION

This patent application claims the benefit and priority of Chinese Patent Application No. 202310632792.3, filed with the China National Intellectual Property Administration on May 31, 2023, the disclosure of which is incorporated by reference herein in its entirety as part of the present application.

### REFERENCE TO SEQUENCE LISTING

A computer readable XML file entitled “GWP20231007656_seqlist”, that was created on Jan. 16, 2024, with a file size of about 30,401 bytes, contains the sequence listing for this application, has been filed with this application, and is hereby incorporated by reference in its entirety.

### TECHNICAL FIELD

The present disclosure belongs to the technical field of viral genetic engineering, and specifically relates to a pseudorabies virus (PRV) expressing a porcine circovirus 2 (PCV2) capsid protein on an envelope, and use thereof.

### BACKGROUND

Pseudorabies (PR), also known as Aujeszky's disease, is an acute animal infectious disease caused by pseudorabies virus (PRV), a member of the subfamily Alphaherpesvirinae in the family Herpesviridae, and has caused serious losses to the pig breeding industry in many countries around the world. At present, during the pig breeding, pigs need to be immunized multiple times to achieve immune protection against different pathogens. The personnel movement in the pig pen and the immune process during immunization cause immune stress in pigs, leading to increased body temperature, anorexia, metabolic changes, slowed growth, and extended feeding cycles. Moreover, multiple immunizations may increase labor costs. Therefore, reducing the number of vaccinations has obvious advantages for pig breeding. To achieve this goal, it is of great value and practical significance to the pig breeding industry by the development of bivalent or polyvalent recombinant virus vaccines (namely one vaccine expressing two or more protective antigens against relative pathogens).

The PRV shows typical structural characteristics of herpes viruses. PRV virus particles are spherical, with a diameter of about 180 nm, and are mainly composed of an envelope, a capsid, and a core. The core is mainly composed of proteins wrapped with double-stranded DNAs, and has a shell in an icosahedral structure. In addition, each of the PRV virus particles has a spike structure formed by glycoprotein on its surface. The genome of PRV is composed of a double-stranded DNA, with a size of about 150 kb, and has at least 70 open reading frames (ORFs), which can encode 70 to 100 proteins, such as 11 envelope proteins, 6 of which are non-essential proteins for PRV replication. Additionally, thymidine kinase (TK), encoded by a UL23 gene and serves as an early gene of the PRV, is not necessary for virus replication and is also one of the main virulence proteins of PRV. A gene virulence of the TK-deletion strain is significantly lower than that of a wild-type, such that TK gene is generally used as the first choice for constructing attenuated strain vaccines. As a result, PRV itself has a larger genome that can accommodate larger exogenous gene fragments, and contains multiple non-essential proteins that can be used as exogenous gene insertion sites, making the PRV a desirable viral vector for bi-or polyvalent vaccines. For example, Ben Peeters et al. reported in 1997 that a gD gene of the PRV was replaced with an expression clement containing an independently-expressed classical swine fever E2 gene, thereby constructing a binary recombinant virus. However, the current bi-or polyvalent vaccines using PRV as a carrier are prepared by: replacing the original genes of PRV with exogenous protective antigen genes; alternatively, conducting frameshift inactivation on the original PRV gene, and inserting the exogenous antigen gene; alternatively, inserting the exogenous genes directly into the PRV genome. These exogenous genes can express corresponding proteins in host cells to exert a certain immune protective effect. However, these bi-or polyvalent vaccine requires live vaccines to exert the immune protective effect. That is, host cells need to be infected by the live vaccines, and the exogenous proteins inserted into the viral genome are expressed in the host cells, such that the host body is stimulated to produce antibodies and corresponding immune protection against related viruses. Compared with virus particles directly carrying exogenous proteins, the immune protection in this way has a certain lag. Only when the virus expresses exogenous antigen proteins after replicating in the host target cells can the exogenous antigen stimulate the host to produce immune recognition and initiate an immune response. Since these exogenous antigen proteins are not assembled on virus particles (such as envelopes), these inactivated recombinant PRV particles cannot exert the expected bi-or polyvalent protective effects.

In addition, the existing recombinant virus construction process generally requires multiple steps to achieve the preparation of recombinant viruses. For example, a transfer vector that introduces an expected sequence is constructed, and then co-transfected with the viral genome, and the transfer vector and the viral genome are subjected to homologous recombination to construct a recombinant virus. This is a classic construction method of recombinant virus, but shows low recombination efficiency. Moreover, it is difficult to screen the virus before and after recombination due to the lack of selection markers. Accordingly, it is generally necessary to construct recombinant viruses with selection markers first. For example, in the first round, a recombinant virus carrying an EGFP fluorescent selection marker is constructed through homologous recombination; and in the second round, a transfer vector carrying the target sequence and the recombinant virus carrying the EGFP selection marker undergo homologous recombination. Subsequently, a recombinant virus that causes a cytopathic effect but shows no green fluorescence is selected to obtain the expected recombinant virus. The above method requires two recombination events, and each recombination event has a low efficiency. In addition, it is highly difficult and the screening the non-fluorescent cells from fluorescent background is time-consuming. These are extremely inconvenient for the construction of recombinant viruses.

In summary, PRV has a large genome and is a suitable viral vector for developing bivalent or polyvalent vaccines. However, due to the current complex experimental operations of recombinant viruses or the heavy workload of recombinant virus screening, there is a poor success rate of recombinant virus construction. Moreover, exogenous immunogens only exist in the viral genome and are not expressed on PRV particles. Objectively, there is an urgent need for a construction method with simple operations, few links, easy screening, and high success rate for constructing a recombinant virus with exogenous immunogen expressed on a viral envelope. In this way, a rescued virus is not only suitable as an attenuated vaccine, but also an inactivated vaccine with a high biological safety. The rescue of the above recombinant viruses can help accelerate the development and application of bivalent and polyvalent vaccines using PRV as a viral vector to serve the pig breeding industry.

### SUMMARY

In view of this, an objective of the present disclosure is to provide a pseudorabies virus (PRV) expressing a porcine circovirus (PCV) capsid protein on an envelope. In the present disclosure, one or more exogenous immunogens and PRV autoimmunogens are expressed on viral particles. When the viral particles are recognized by the body's immune system, the host's immune system simultaneously initiates recognition of all immunogens on the viral particles (including exogenous immunogens and PRV autoimmunogens) and initiates an immune response. Therefore, a protective effect of the bi-or polyvalent vaccine based on PRV particles can be better exerted to avoid the disadvantage of conventional vaccines that require multiple immunizations of animals due to single disease prevention.

To achieve the above objective, the present disclosure provides the following technical solutions:

The present disclosure provides a recombinant PRV particle expressing an exogenous immunogen on an envelope, where the recombinant PRV particle uses a gene corresponding to a non-essential envelope protein of a PRV as an insertion site, and replaces a gene sequence corresponding to an amino acid in an extramembrane domain of the non-essential envelope protein with a coding sequence corresponding to the exogenous immunogen.

Preferably, the exogenous immunogen is derived from a pathogen.

Preferably, the exogenous immunogen is derived from PCV2.

Preferably, the recombinant PRV particle uses a gE gene of the PRV as the insertion site; the gE gene includes a gE initiation codon and a cytomegalovirus (CMV) promoter before the gE initiation codon;

a modified enhanced green fluorescent protein (EGFP) gene is ligated after a gE termination codon of the gE gene, and two loxP sites in a same direction are inserted at both ends of the modified EGFP gene.

The present disclosure further provides a transfer vector of the recombinant PRV particle.

The present disclosure further provides a construction method of a recombinant PRV with green fluorescence, including the following steps: subjecting a eukaryotic cell to co-transfection using the transfer vector and a genome of an attenuated PRV vaccine strain, and collecting a co-transfection mixture that has a lesion and EGFP green fluorescence; and

centrifuging the co-transfection mixture to collect a supernatant, and conducting plaque purification using the supernatant to obtain the recombinant PRV with green fluorescence.

The present disclosure further provides a recombinant PRV with green fluorescence constructed by the construction method.

The present disclosure further provides a construction method of a recombinant PRV without fluorescence, including the following steps: extracting a genome of the recombinant PRV with green fluorescence, and mixing the genome with a Cre enzyme to allow enzymatic digestion in vitro; transfecting a resulting enzymatic digestion product into a eukaryotic cell, and collecting a co-transfection mixture that has a lesion but no fluorescence; selecting a clone with no fluorescence but showing the lesion by conducting plaque screening on the co-transfection mixture to obtain the recombinant PRV without fluorescence.

The present disclosure further provides a recombinant PRV without fluorescence constructed by the construction method.

The present disclosure further provides use of the transfer vector or the recombinant PRV without fluorescence in preparation of a bivalent vaccine or a polyvalent vaccine based on PRV/circovirus (CV).

Beneficial effects: the present disclosure provides a recombinant PRV particle expressing an exogenous immunogen on an envelope. In the present disclosure, an attenuated PRV vaccine strain is used as a vector to express an exogenous immunogen. The exogenous immunogen only replaces an extracellular domain of a non-essential envelope protein of a PRV, and retains a transmembrane domain and an intracellular domain of an original envelope protein, such that one or more exogenous immunogens and PRV autoimmunogens are expressed on virus particles. When the virus particles are recognized by the body's immune system, the host's immune system simultaneously starts to recognize all immunogens on the virus particles and initiate an immune response, thereby exerting the protective effect of the bi-or polyvalent vaccine based on PRV virus particles.

The present disclosure provides a fast and efficient virus rescue method that requires only one recombination, which is different from the classic two recombination methods. Only one homologous recombination is needed to achieve rescue of the recombinant virus, and “what you see is what you need.” As long as EGFP fluorescence and cellular lesions appear, it means the virus rescue is successful. The cells expressing EGFP are selected from non-fluorescent background with high screening speed and efficiency. This avoids two recombinations and the need to screen out fluorescent cells from a background of fluorescent cells during the second homologous recombination, which is extremely difficult when there is no fluorescence but cell lesions occur.

If it is necessary to remove selection markers such as EGFP, in vitro biochemical enzymatic digestion is conducted to remove selection marker sequences such as EGFP. This method has an enzymatic digestion efficiency of up to 50%, making the subsequent screening of rescue viruses without screening markers extremely successful. The screening is extremely convenient, and pure recombinant viruses can be obtained starting from the second generation. This method completely avoids the low DNA transfection efficiency or unstable transfection during the second recombination of traditional methods. In an example, the extracellular domain of the virus particle is the first domain that comes into contact with the body's immune cells, and the extracellular domain of gE is replaced with the exogenous immunogen, PCV2 Cap protein. The exogenous protein is expressed on the outside of an envelope membrane of the virus particle, while the transmembrane domain and intracellular domain remain unchanged, allowing the envelope to carry the exogenous immunogen while maintaining the original state of the virus particle to the greatest extent. This achieves duplexing immunization at the virus particle level, rather than just using PRV as a gene carrier for exogenous antigens, making the final exogenous protein produced independent of virus particles.

### BRIEF DESCRIPTION OF THE DRAWINGS

FIG. 1 shows signal peptide prediction results of the gE protein;

FIG. 2 shows prediction results of the extramembrane domain, transmembrane domain, and intramembrane domain of the gE protein;

FIG. 3 shows a construction strategy of the recombinant transfer vector;

FIGS. 4A-B show that a rescued virus PRV TK⁻/gE⁻/PCV2 dCap⁺/EGFP⁺ shows green fluorescence and cytopathic changes at the same time;

FIG. 5 shows PCR verification results of the presence of insertion sequences at a gE site of different generations of a rescue virus PRV TK⁻/gE⁻/PCV2 dCap⁺ with the EGFP fluorescent marker removed;

FIG. 6 shows WB verification results of dCap protein expression of different generations of the rescue virus PRV TK⁻/gE⁻/PCV2 dCap⁺ with the EGFP fluorescent marker removed;

FIG. 7 shows IFA verification results of dCap protein expression of different generations of the rescue virus PRV TK⁻/gE⁻/PCV2 dCap⁺ with the EGFP fluorescent marker removed;

FIG. 8 shows a one-step growth curve of the rescue virus PRV TK⁻/gE⁻/PCV2 dCap⁺;

FIG. 9 shows immunoelectron microscopy results of the recombinant virus PRV TK⁻/gE⁻/PCV2 dCap⁺;

FIGS. 10A-B show production of antibodies against PRV gB and PCV2 dCap proteins by the recombinant virus PRV TK⁻/gE⁻/PCV2 dCap⁺ after immunizing mice 7 d, 14 d, and 21 d;

FIGS. 11A-B show survival curves of mice challenged with the recombinant virus PRV TK⁻/gE⁻/PCV2 dCap⁺ after immunization; and

FIGS. 12A-B show neutralizing antibody titers against PRV and PCV2 produced by the recombinant virus PRV TK⁻/gE⁻/PCV2 dCap⁺ in mice after immunization.

### DETAILED DESCRIPTION OF THE EMBODIMENTS

The present disclosure provides a recombinant PRV particle expressing an exogenous immunogen on an envelope, where the recombinant PRV particle uses a gene corresponding to a non-essential envelope protein of a PRV as an insertion site, and replaces a gene sequence corresponding to an amino acid in an extramembrane domain of the non-essential envelope protein with a coding sequence corresponding to the exogenous immunogen.

In the present disclosure, the exogenous immunogen may be one type or multiple types; the exogenous immunogen may be from a pathogen, preferably a virus that causes swine viral diseases. In the examples, PCV2 is preferably used as the exogenous immunogen for explanation, but cannot be regarded as the entire protection scope of the present disclosure.

In the present disclosure, the PRV preferably includes the PRV genotype II gE⁻TK⁻ double gene-deletion vaccine strain that broke out after 2011 (PRV HD/c strain, NCBI serial number MZ063026, patent number ZL201710774869.5) as a vaccine vector to express the immunogen of PCV2. This immunogen only replaces the extracellular domain of the non-essential envelope protein of PRV and retains the transmembrane domain and intracellular domain of the original envelope protein. More preferably, amino acid sequences of a gE gene (SEQ ID NO: 3 is the insertion site, while retaining the gE signal peptide (amino acids 1 to 23)), a transmembrane domain (amino acids 431 to 453), and an intramembrane domain (amino acids 454 to 579) of a PRV DX strain (NCBI sequence number MZ063026.1) are selected. Meanwhile, only the nucleotide sequence corresponding to the amino acids in the extramembrane domain (amino acids 24 to 430) is replaced with the PCV2 Cap gene sequence (dCap, SEQ ID NO: 1) without the nuclear localization signal, and the dCap protein translated by the nucleotides has a sequence shown in SEQ ID NO: 2. The virus particle uses the gE gene of PRV as the insertion site, and inserts a high-efficiency exogenous promoter before the gE initiation codon, preferably a CMV promoter, to enhance the expression of the target gene PCV2 dCap; a modified EGFP gene is ligated after the gE termination codon, two loxP sites in a same direction are inserted at both ends of the EGFP gene, and the recombinant virus is selected with the EGFP as a screening marker. On this basis, upstream 1081 bp and downstream 1246 bp sequences of the gE gene are used as a transfer vector and homologous left and right arms of the PRV genome to form the transfer vector of the present disclosure: SEQ ID NO: 4: left arm (1-1081 bp)-CMV promoter (1082-1670 bp)-Kozak sequence (1671-1676 bp)-gE signal peptide related sequence (1677-1805 bp)-PCV2 dCap (1806-2381 bp)-gE transmembrane+intramembrane (2382-2930 bp)-loxP (2931-2964 bp)-modified EGFP (2965-4269 bp)-loxP (4270-4303 bp)-right arm (4304-5549 bp).

The present disclosure further provides a transfer vector of the recombinant PRV particle.

In the present disclosure, after constructing the recombinant PRV particle, primers are preferably designed for each fragment of the corresponding genome sequence of the recombinant PRV particle (Table 1). Each fragment is spliced using homologous recombination kits and other methods, and finally constructed into a pUC18 vector to obtain the pUC18-CMV/gE-PCV2 dCap⁺/EGFP⁺ transfer vector.

TABLE 1
Primer sequences of transfer vectors

| | | SEQ | |
|---|---|---|---|
| | | ID | Target |
| Primer name | Sequence (5′-3′) | NO: | fragment |
| LgE-F-pUC18 | GAATTCGAGCTCGGTACCCACGTCGCCGGCAG | 5 | Homologous |
| plus | CGCCGTCCTC | | left arm |
| LgE-R-CMV | TTGATTACTATTAATAACTAGGTCTCAACCCC | 6 | |
| plus | GGTGTGTG | | |
| CMV-F-LgE | CACACACCGGGGTTGAGACCTAGTTATTAATA | 7 | CMV promoter |
| plus | GTAATCAATTAC | | |
| CMV-R-gESP | CGCAGCAGAAAGGGCCGCAT | 8 | |
| plus | GGTGGCGATCTGACGGTTCACTAAACCAG | | |
| gESP-F-CMV | GTGAACCGTCAGATCGCCACCATGCGGCCCTT | 9 | gE signal |
| plus | TCTGCTGC | | peptide |
| gESP-R-2dCap | CGGGTGTTGAAGATGCCATTGGCCGAGGGACT | 10 | |
| plus | CGGGACCTCGGTGAC | | |
| 2dCap-F-gESP | AGGTCCCGAGTCCCTCGGCCAATGGCATCTTC | 11 | PCV2 dCap |
| plus | AACACCCGCCTCTC | | |
| 2dCap-R-gEKB | ATCGCGTCGTCGCCGCCGCCAGGGTTAAGTGG | 12 | |
| plus | GGGGTCTTTAAG | | |
| gEKB-F-2dCap | AAGACCCCCCACTTAACCCTGGGGGGGGCGAC | 13 | gE |
| plus | GACGCGATCTAC | | transmembrane |
| gEKB-R-PUC18 | GACGGCCAGTGCCAAGCTTTTAAGCGGGGGGG | 14 | domain + |
| plus | GACATCAACAG | | intramembrane |
| gEKB-R-loxP | ATAACTTCGTATAGCATACATTATACGAAGTT | 15 | domain |
| plus | ATTTAAGCGGGGGGGGACA | | |
| EGFP-F | TGTATGCTATACGAAGTTATGCCCCTCTCCCT | 16 | EGFP sequence |
| | CCCCCCCCCCTAA | | |
| EGFP-R-loxP | ATAACTTCGTATAGCATACATTATACGAAGTT | 17 | |
| plus | ATCTACTTGTACAGCTC | | |
| RgE-F-loxP | TGTATGCTATACGAAGTTATATACCGGGAGAA | 18 | Homologous |
| plus | CCGGTG | | right arm |
| RgE-R-pUC18 | GACGGCCAGTGCCAAGCTTGTTGTGGACCCGC | 19 | |
| plus | GCGAACATGGCG | | |

The present disclosure further provides a construction method of a recombinant PRV with green fluorescence, including the following steps: subjecting a eukaryotic cell 239T to co-transfection using the transfer vector and a genome of an attenuated PRV vaccine strain, and collecting a co-transfection mixture that has a lesion and EGFP green fluorescence; and

centrifuging the co-transfection mixture to collect a supernatant, and conducting plaque purification using the supernatant to obtain the recombinant PRV with green fluorescence.

In the present disclosure, the transfer vector is preferably co-transfected into 293T cells together with the genome of the PRV II gE⁻TK⁻ vaccine strain (PRV HD/c strain). Homologous recombination between the transfer vector and the viral genome is achieved through homologous arms, and the segment in the middle of the homologous arm on the transfer vector is replaced with the corresponding gene of the virus. The cells and supernatant that have lesions and EGFP fluorescence are collected, centrifuged at 12,000 g to obtain a supernatant, which is preferably diluted 10, 10², 10³, 10⁴, 10⁵, and 10⁶ times for plaque purification. A pure PRV TK⁻/gE⁻/PCV2 dCap⁺/EGFP⁺ recombinant PRV with green fluorescence is obtained after a total of 5 rounds of plaque purification.

The present disclosure further provides a recombinant PRV with green fluorescence constructed by the construction method.

The present disclosure further provides a construction method of a recombinant PRV without fluorescence, including the following steps: extracting a genome of the recombinant PRV with green fluorescence, and mixing the genome with a Cre enzyme to allow enzymatic digestion; transfecting a resulting enzymatic digestion product into a eukaryotic cell, and collecting a co-transfection mixture that has a lesion but no fluorescence; selecting a clone with no fluorescence but showing the lesion by conducting plaque screening on the co-transfection mixture to obtain the recombinant PRV without fluorescence.

In the present disclosure, in order to delete the selection marker, the genome of the PRV TK⁻/gE⁻/PCV2 dCap⁺/EGFP⁺ recombinant PRV is preferably extracted by conventional methods; the EGFP fluorescent expression element is removed by in vitro enzymatic digestion with Cre enzyme for 2 h, and a Cre enzyme-treated genome is transfected into eukaryotic cells using the same method as above; clones that show no fluorescence and lesions are selected through plaques to obtain PRV TK⁻ /gE⁻/PCV2 dCap⁺ recombinant PRV.

The present disclosure further provides a recombinant PRV without fluorescence constructed by the construction method.

In the recombinant PRV without fluorescence (PRV TK⁻/gE⁻/PCV2 dCap⁺) obtained by the construction method of the present disclosure, the EGFP has been removed, and the dCap protein can be expressed smoothly. This indicates that the recombinant PRV is a potentially desirable bivalent vaccine carrier and can be used to prepare PRV-based bivalent vaccines or polyvalent vaccines.

The present disclosure further provides use of the transfer vector or the recombinant PRV without fluorescence in preparation of a bivalent vaccine or a polyvalent vaccine based on PRV/circovirus (CV).

The PRV expressing a PCV2 capsid protein on an envelope, and use thereof provided by the present disclosure are described in detail below with reference to the examples, but these examples should not be understood as limiting the claimed scope of the present disclosure.

### Example 1

Rescue of recombinant PRV expressing PCV2 viral Cap protein on PRV TK⁻/gE⁻ viral envelope

1. Construction of recombinant transfer vector

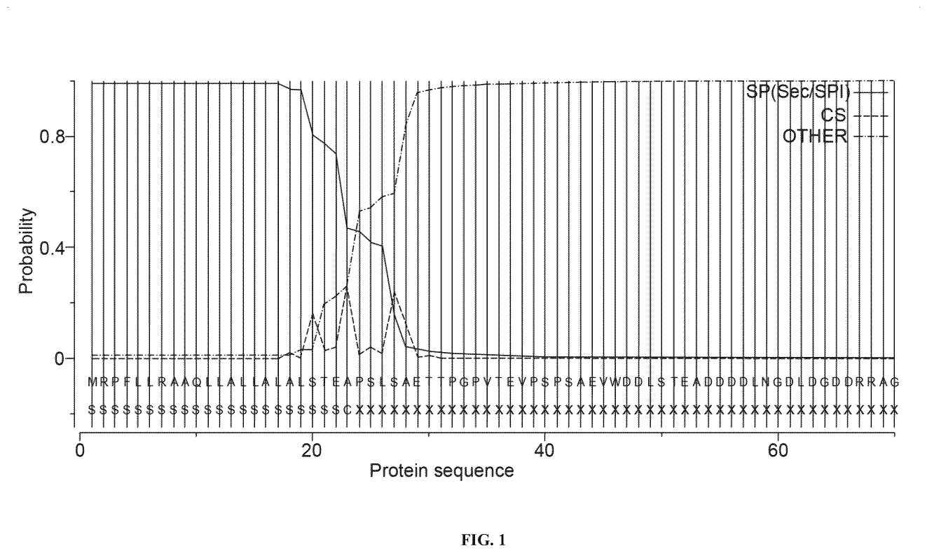

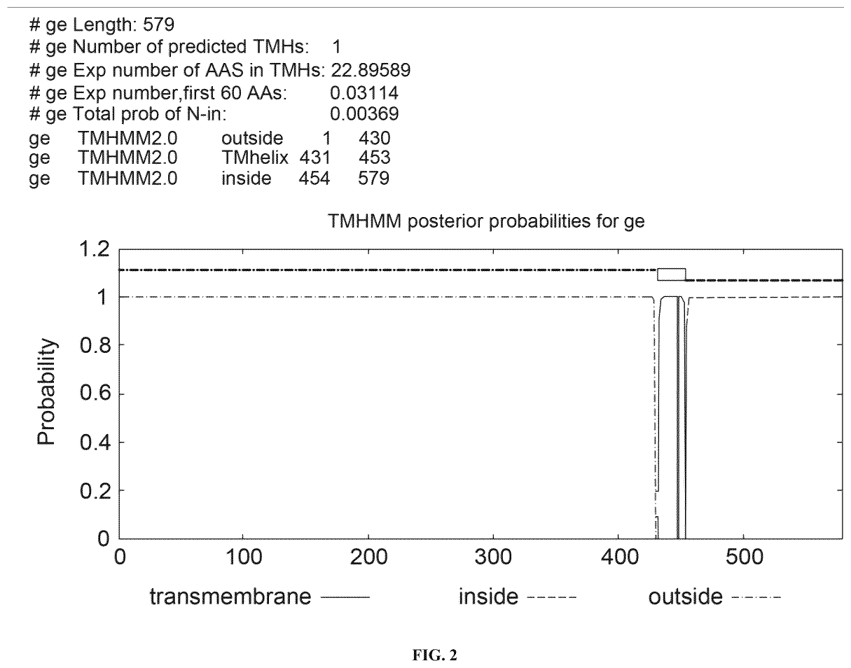

The signal peptide (FIG. 1) as well as extramembrane domain, transmembrane domain, and intramembrane domain (FIG. 2) of PRV DX strain gE protein were predicted using online software SignalP-5.0 Server and TMHMM Server v.2.0, respectively.

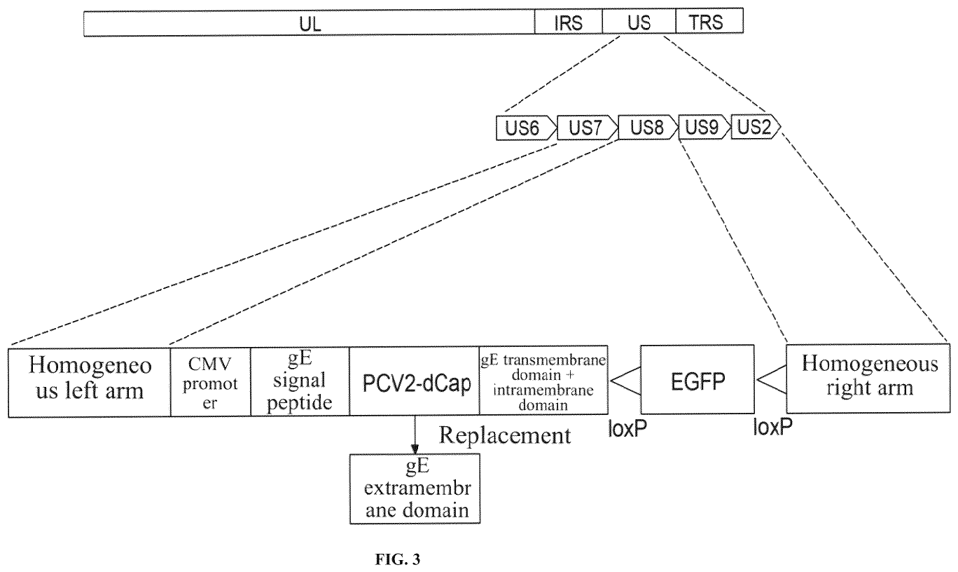

A specific construction strategy of the transfer vector included: gE (US8 gene) was selected as an insertion site, and a CMV promoter was added before a gE signal peptide (i.e, an initiation codon ATG) to enhance the expression of a target gene PCV2 dCap; a modified EGFP gene carrying loxP sites in a same direction at both ends was ligated after a gE termination codon TAA; upstream 1,081 bp and downstream 1,246 bp gene fragments of the gE serve as the homologous left and right arms required for homologous recombination between the vector and the PRV genome. Primers were designed for each fragment of the corresponding genome sequence, and an expected sequence was amplified using a PRV DX strain (NCBI sequence number: MZ063026.1) genome as a template; each fragment was spliced using a homologous recombination kit, and finally constructed into the pUC18 vector to obtain a pUC18-CMV/gE⁻/PCV2 dCap⁺/EGFP⁺ transfer vector (FIG. 3). A DNA fragment containing 1,081 bp upstream of gE, the complete gE gene, and 1,246 bp downstream of gE was still amplified using the PRV DX strain genome as a template and constructed into the pUC18 vector as a control transfer vector.

2. Extraction of PRV HD/c genome

The PRV HD/c strain was inoculated into a 100 mm² cell culture dish filled with a single layer of Vero cells at a ratio of 1:1000, and the viral DNA was extracted by the following steps when the cells showed 80% lesion shrinkage (about 24 h):

(1) the virus solution and cells were collected in the cell flask, added with 1 mL of a cell lysis solution, then added with proteinase K (20 mg/mL) to a final concentration of 0.2 mg/mL, mixed by vortexing well, and placed in a 55° C. water bath for 30 min;

(2) an equal volume of a mixture of phenol: chloroform=25:24 was added, mixed well by shaking vigorously, and centrifuged at 12,000 rpm at 4° C. for 10 min to obtain a supernatant;

(3) 2 times a volume of absolute ethanol pre-cooled at −20° C. was added, mixed well by inverting, precipitated and allowed to stand for 20 min at −20° C., centrifuged at 12,000 rpm for 10 min at 4° C., and a resulting supernatant was discarded;

(4) the remaining precipitate was washed with 1 mL of 75% ethanol, centrifuged at 12,000 rpm for 5 min at 4° C., and a resulting supernatant was discarded; and

(5) the remaining precipitate containing viral genomic DNA was dried, dissolved with an appropriate volume of TE (containing RNase), a small amount of an obtained DNA solution was collected for concentration determination, and then stored at −20° C.

3. Rescue of TK⁻/gE⁻/PCV2 dCap⁺/EGFP⁺ recombinant PRV

3.1. Transfection experiment (single well of a six-well plate) with a BioBEST transfection reagent:

(1) the following components were added to a sterile 1.5 mL centrifuge tube:

1 μg of viral genomic DNA and 2 μg of transfer vector, 1 μg of viral genomic DNA (positive control), and 2 μg of transfer vector (negative control);

(2) 400 μL of serum-free DMEM medium was added, mixed gently by pipetting and allowed to stand at room temperature for 2 min;

(3) 8 μL of transfection reagent was added, mixed well by gently pipetting, and then allowed to stand at room temperature for 15 min;

(4) a resulting mixture was added dropwise into the cell wells, mixed well by shaking gently, and then incubated in a 37° C. incubator;

(5) after 6 h of incubation, the medium containing the transfection reagent was added, replaced with a new medium (2 mL of DMEM containing 2% FBS), incubated in a 37° C. incubator, while the cell status was observed every day (green fluorescence was seen in the well transfected with the transfer vector);

(6) after lesions appeared on the 293T cells, the cultured supernatant and cells were harvested in all wells, frozen and thawed 1 time at −70° C., centrifuged at 12,000 rpm for 10 min at 4° C. to obtain a supernatant, which was inoculated into a confluent monolayer of Vero cells in a 96-well plate, incubated at 37° C. for 1 h, a supernatant was discarded, and the cells were transferred to a medium containing 2% FBS to continue the culturing; and

(7) When only cytopathic changes were seen in the wells infected with the single-transfected viral genome, while both cytopathic changes and green fluorescence were observed in the wells infected with the co-transformed viral genome and the transfer vector, a lesioned well with green fluorescence was selected to collect the virus solution, which was frozen and thawed 1 time at −70° C., centrifuged at 12,000 rpm for 10 min at 4° C., and a supernatant was collected.

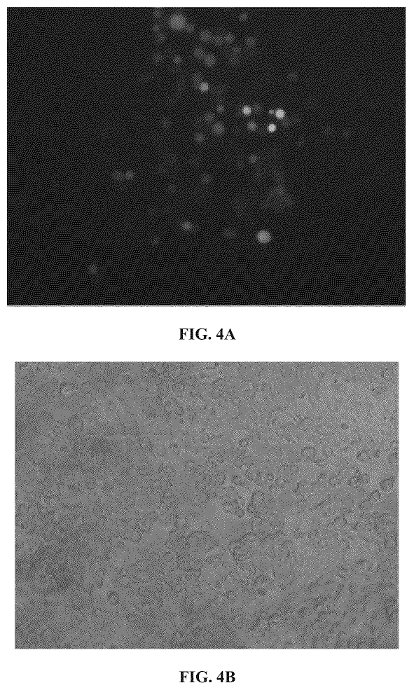

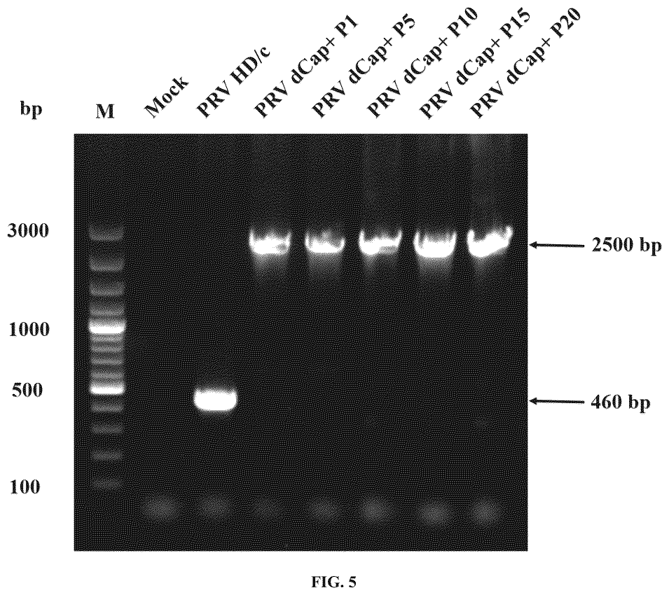

The results were shown in FIGS. 4A-B. The PRV TK⁻/gE⁻/PCV2 dCap⁺/EGFP⁺ appeared green fluorescence and cytopathic changes at the same time, indicating that the virus rescue was successfully completed.

3.2. Plaque purification of recombinant PRV TK⁻/gE⁻/PCV2 dCap⁺/EGFP⁺

(1) the virus solution was diluted 10 times (10⁻¹ to 10⁻⁶) and inoculated into a six-well plate filled with Vero cells, at 100 μL/well, and incubated at 37° C. for 1 h;

(2) a cultured supernatant was discarded, an equal volume of mixed 2× DMEM medium (containing 2% FBS) and 2% low-melting point agarose were added, solidified, placed upside down, and cultured in a 37° C. incubator for 36 h to 48 h to observe visible white dots under light, known as plaques;

(3) the plaques with green fluorescence were observed and marked under a fluorescence microscope, the plaques were selected using a sterilized yellow pipette tip, pipetted several times in a DMEM culture medium, frozen and thawed 1 time at −70° C. to collect the virus; and

(4) steps (1) to (3) were repeated for plaque purification of the mixed virus containing both PRV HD/c and recombinant PRV TK⁻/gE⁻/PCV2 dCap⁺/EGFP⁺ until all plaques showed green fluorescence and no HD/c genome detected, such that the PRV TK⁻/gE⁻/PCV2 dCap⁺/EGFP⁺ recombinant virus was obtained.

The recombinant PRV TK⁻/gE⁻/PCV2 dCap⁺/EGFP⁺ was inoculated into a 75 cm² cell flask filled with Vero cells. When the cells showed 80% cytopathic effects, the genome of the recombinant virus was extracted using the aforementioned method.

4. Rescue of recombinant PRV TK⁻/gE⁻/PCV2 dCap⁺

4.1 Excision of EGFP gene by enzymatic digestion using Cre recombinase

(1) reaction system: 5 μL of 10×Cre recombinase reaction buffer, 10 μg of viral genomic DNA, 1 μL of Cre recombinase, 50 μL of ddH₂O;

reaction conditions: 37° C. for 30 min; 70° C. for 10 min (thermal inactivation);

(2) a resulting reaction product was added with 200 μL of a mixture of phenol: chloroform=25:24, mixed well by inverting, and centrifuged at 12,000 rpm at 4° C. for 15 min to a supernatant;

(3) pre-cooled absolute ethanol (2 times the volume) was added, mixed well by inverting, precipitated and allowed to stand for 20 min at −20° C., centrifuged at 12,000 rpm for 10 min at 4° C., and a resulting supernatant was discarded;

(4) the remaining precipitate was washed with 500 μL of 75% ethanol, centrifuged at 12,000 rpm for 5 min at 4° C., and a resulting supernatant was discarded; and

(5) the remaining precipitate was air-dried, dissolved in an appropriate volume of TE, a small amount of an obtained DNA solution was collected for concentration determination, and then stored at −20° C.

4.2 Transfection of 293T cells with viral nucleic acid to rescue recombinant PRV TK⁻/gE⁻/PCV2 dCap⁺

The steps were the same as 3.1, where the control transfer vector prepared in step 1 and the PRV TK⁻/gE⁻/PCV2 dCap⁺/EGFP⁺ genome were co-transfected to obtain a control group. The results showed that when the genome obtained by the Cre recombinant enzymatic digestion method was transfected, about 50% of the areas where cell lesions appeared had no green fluorescence. The wells that had no green fluorescence but showed cytopathic effects were selected, and the virus solution in the wells was collected for plaque purification. In the control group, only 2% of the cells showed no green fluorescence. This indicated that the traditional homologous recombination method to eliminate the EGFP selection marker had low recombination efficiency, and the method used in the present disclosure could greatly improve the recombination efficiency.

4.3. Plaque purification of recombinant PRV TK⁻/gE⁻/PCV2 dCap⁺

The method was the same as that in step 3.2, except that the plaques without green fluorescence were observed and marked under a fluorescence microscope, the plaques were selected with a yellow pipette tip, pipetted in DMEM culture medium several times, and frozen and thawed 1 time at −70° C.; the plaque purification was continuously conducted according to the above method until all plaques showed no green fluorescence.

4.4. Identification of recombinant PRV TK⁻/gE⁻/PCV2 dCap⁺ strain

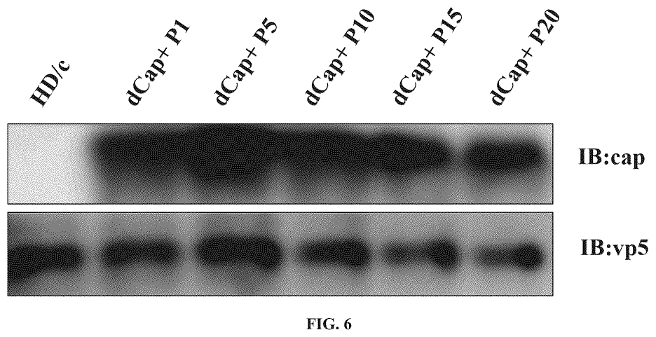

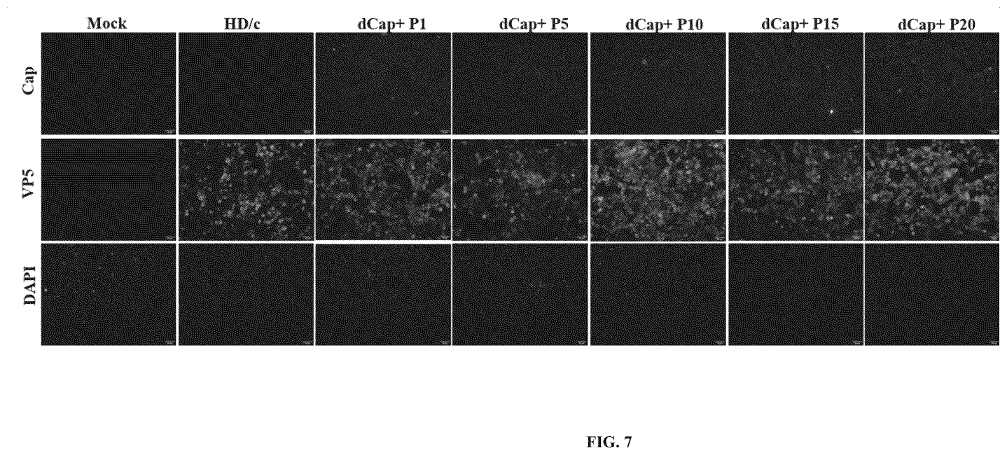

The upstream and downstream genes of gE were PCR-amplified with specific primers (SEQ ID NO: 20, gE-US7-9 F: ATCTTCCTGGGCGGGATCGCCT; SEQ ID NO: 21, gE-US7-9 R: AGATGACCAGCGCGGCGGCGCTGAT) to detect the presence of PCV2 dCap gene. The conventional Western blot (WB) (the supernatant of each generation of PRV TK⁻/gE⁻/PCV2 dCap⁺ and PRV HD/c control viruses were ultrafiltrated and centrifuged to collect the concentrated virus solution, protein samples were prepared by conventional methods, and then detected by WB using VP5 and Cap antibodies) and indirect immunofluorescence (the PRV TK⁻/gE⁻/PCV2 dCap⁺ and PRV HD/c control virus supernatants of each passage were inoculated into PK15 cells at a density of 80% and MOI=1, fixated with 4% paraformaldehyde at 4° C. for 30 min at 24 hpi, blocked with 5% skim milk for 30 min, and incubated with VP5 (1:400) and Cap (1:400) as primary antibodies at 37° C. for 2 h. The cells were washed three times with PBS and incubated with FITC (1:400) and A546 working solution as secondary antibodies for 45 min. After 5 min of DAPI (1:5000) treatment, fluorescence was observed to identify whether the recombinant PRV TK⁻/gE⁻/PCV2 dCap⁺-infected cells expressed the PCV2 dCap). The results were shown in FIG. 5 to FIG. 7. EGFP had been removed, Cap protein could be expressed smoothly, and PCR, WB, and IFA results were not significantly different from those of the P5 generation even after being passaged to the P20 generation. This indicated that the recombinant PRV could stably express exogenous PCV2 dCap, and that the rescued recombinant virus could be used as a potential virus species for bivalent vaccines.

4.5 Drawing of one-step growth curve

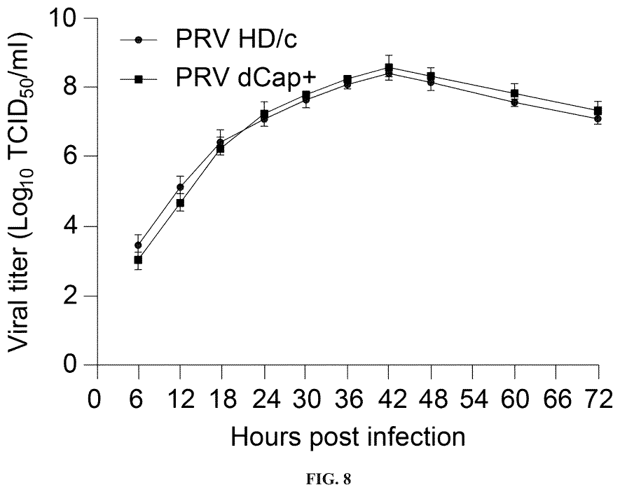

PRV HD/c strain and PRV TK⁻/gE⁻/PCV2 dCap⁺ strain were continuously passaged in PK15 cells to a P10 generation. A virus supernatant of the P10 generation with Moi=1 was inoculated with PK15 cells in a 6-well plate with a cell monolayer density of 90%, incubated at 37° C. for 2 h, rinsed three times with DMEM, then transferred to a 2% FBS DMEM and cultured in the incubator. The cells and supernatant were scraped and collected at 4 hpi, 8 hpi, 12 hpi, 18 hpi, 24 hpi, 30 hpi, 36 hpi, 48 hpi, 60 hpi, and 72 hpi, frozen and thawed at −80° C. 1 time and then centrifuged at 12,000 rpm for 5 min. The virus supernatant was taken to measure TCID₅₀, and the corresponding one-step growth curve was drawn by GraphPad (FIG. 8). The results showed that the growth of the recombinant virus was no different from that of the parent strain.

4.6 Immunoelectron microscopy

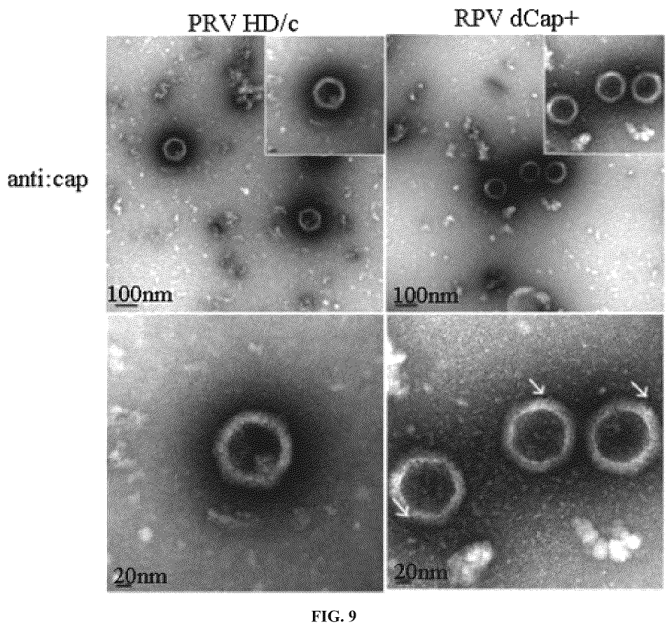

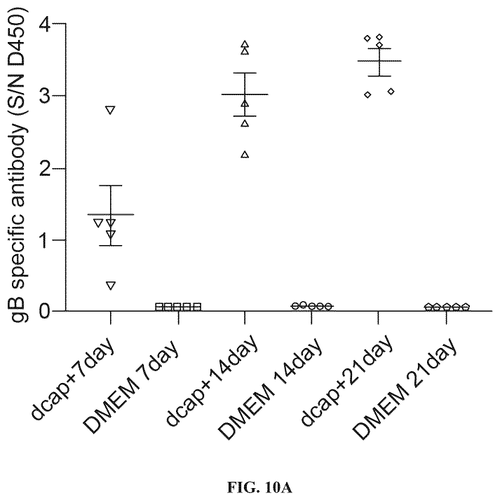

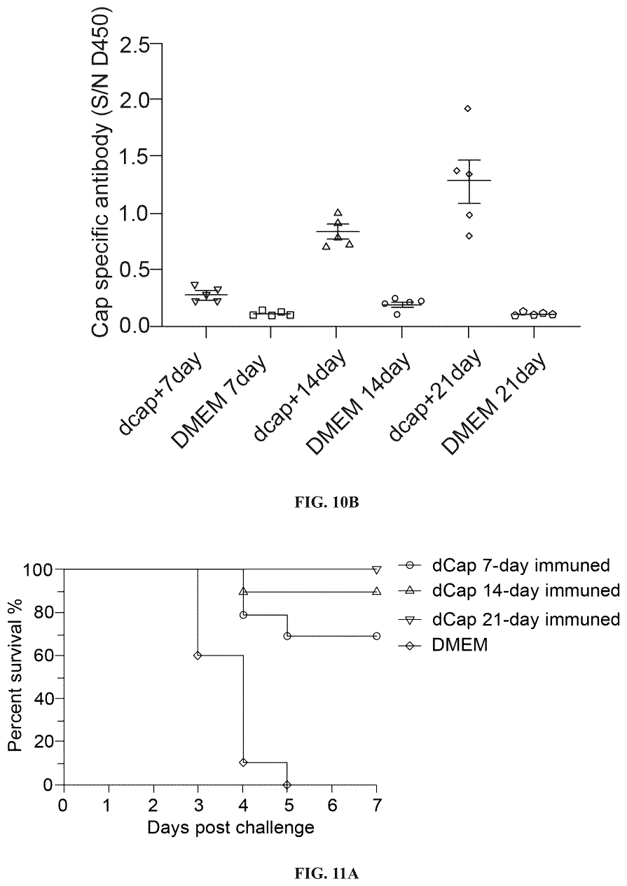

This experiment was conducted with reference to a method of “Freezing Microscopic Immunolabeling Technology” (Huang Bingquan, Chemical Industry Press, 2007). The results of immunoelectron microscopy were shown in FIG. 9, indicating that the PCV2 dCap protein stably existed on the envelope of the PRV.

5. Antibody production in PRV TK⁻/gE⁻/PCV2 dCap⁺-immunized mice after 7 d, 14 d, and 21 d

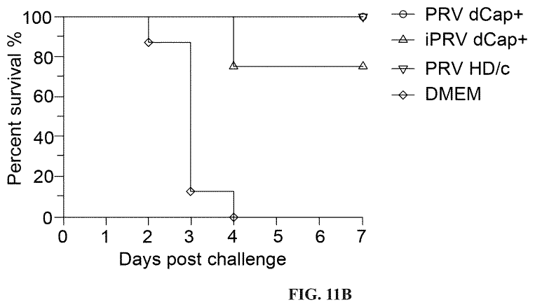

Balb/c mice were immunized with 0.1 mL of PRV TK⁻/gE⁻/PCV2 dCap⁺ virus dilution (containing 7.5 TCID₅₀), while a DMEM control group was established. The serum of mice in each group was collected from the infraorbital venous plexus on 7 d, 14 d, and 21 d after vaccination, and the mouse serum ELISA titers were detected using commercial PRV gB antibody kit and PCV2 Cap antibody kit. The results were shown in FIGS. 10A-B. With the prolongation of immunization time, the antibody titers against PRV gB and PCV2 Cap gradually increased, while the titers in the DMEM control group were always at the baseline level. This indicated that the PRV TK⁻/gE⁻/PCV2 dCap⁺ could effectively stimulate mice to produce antibodies against PRV and PCV2 simultaneously. IFA testing also showed similar results (Tables 2 and 3).

TABLE 2
IFA titers of PRV antibodies in PRV TK⁻/gE⁻/PCV2
dCap⁺-immunized mice after 7 d, 14 d, and 21 d

| | | IFA titers of PRV antibodies | | |
|---|---|---|---|---|
| | | in PRV TK⁻/gE⁻/PCV2 dCap⁺- | | |
| | | immunized mice after 7 d, 14 d, and 21 d | | |
| Group | | 7 d | 14 d | 21 d |
| PRV dCap⁺ | M1 | 1:400 | 1:3200 | 1:3200 |
| | M2 | 1:400 | 1:1600 | 1:3200 |
| | M3 | 1:800 | 1:1600 | 1:3200 |
| | M4 | 1:800 | 1:1600 | 1:1600 |
| | M5 | 1:400 | 1:1600 | 1:1600 |
| DMEM | M1 | <1:100 | <1:100 | <1:100 |
| | M2 | <1:100 | <1:100 | <1:100 |
| | M3 | <1:100 | <1:100 | <1:100 |
| | M4 | <1:100 | <1:100 | <1:100 |
| | M5 | <1:100 | <1:100 | <1:100 |

TABLE 3
IFA titers of PRV2 Cap antibodies in PRV TK⁻/gE⁻/PCV2
dCap⁺-immunized mice after 7 d, 14 d, and 21 d

| | | IFA titers of PRV2 Cap antibodies | | |
|---|---|---|---|---|
| | | in PRV TK⁻/gE⁻/PCV2 dCap⁺- | | |
| | | immunized mice after 7 d, 14 d, and 21 d | | |
| Group | | 7 d | 14 d | 21 d |
| PRV dCap⁺ | M1 | 1:400 | 1:400 | 1:800 |
| | M2 | 1:800 | 1:800 | 1:1600 |
| | M3 | 1:800 | 1:800 | 1:1600 |
| | M4 | 1:800 | 1:400 | 1:800 |
| | M5 | 1:400 | 1:800 | 1:1600 |
| DMEM | M1 | <1:100 | <1:100 | <1:100 |
| | M2 | <1:100 | <1:100 | <1:100 |
| | M3 | <1:100 | <1:100 | <1:100 |
| | M4 | <1:100 | <1:100 | <1:100 |
| | M5 | <1:100 | <1:100 | <1:100 |

6. Animal challenge protection test of PRV TK⁻/gE⁻/PCV2 dCap⁺

Balb/c mice were immunized with 0.1 mL of PRV TK⁻/gE⁻/PCV2 dCap⁺ virus dilution (containing 7.5 TCID₅₀), and lethal doses of PRV DX 0.1 mL (including 5.5 TCID₅₀) were administered on 7 d, 14 d, and 21 d after vaccination to challenge the virus, and the survival rate of mice was recorded.

In order to compare the immune effects of attenuated and inactivated viruses, 0.1 mL of PRV TK⁻/gE⁻/PCV2 dCap⁺ virus dilution (PRV Cap⁺) (containing 7.5 TCID₅₀) and inactivated PRV TK⁻/gE⁻/PCV2 dCap⁺ virus dilution (inactivated, iPRV dCap⁺) (containing 7.5 TCID₅₀) were used to immunize Balb/c mice. At the same time, the PRV HD/c vaccine immunization group was set as a positive control, and the DMEM group was set as a non-immune control. 21 d after inoculation, the mice were challenged with a lethal dose of virulent PRV DX 0.1 mL (containing 5.5 TCID₅₀), and a survival rate of the mice was recorded.

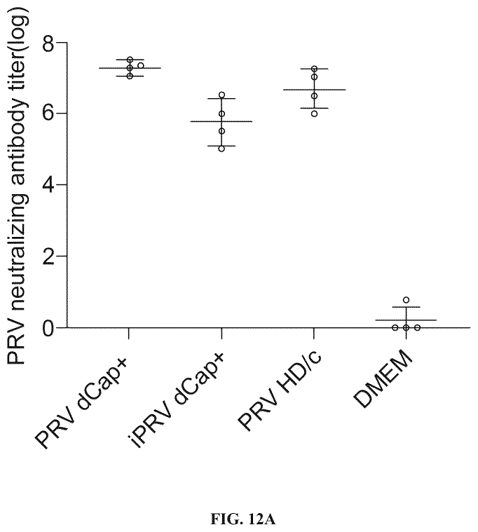

The results were shown in FIGS. 11A-B. PRV TK⁻/gE⁻/PCV2 dCap⁺ was challenged with a lethal dose of PRV DX after 7 d of immunization, and a protection rate was 70%. By the 21st day of immunization, the protection rate reached 100%. When inactivated PRV TK⁻/gE⁻/PCV2 dCap⁺ (inactivated, iPRV dCap⁺) was used for immunization, the protection rate against PRV virulent attack could also reach 70% 21 d after immunization.

7. Neutralizing antibody titers of PRV TK⁻/gE⁻/PCV2 dCap⁺

Four groups: PRV TK⁻/gE⁻/PCV2 dCap⁺ (PRV dCap), inactivated PRV dCap (iPRV dCap), PCV2 inactivated virus (iPCV2) (PCV2 HZ0201NCBI serial number: AY188355.1), and DMEM were used to immunize Balb/c mice for 21 d, each group with 4 biological replicates. Mouse serum was collected from the infraorbital venous plexus, inactivated at 55° C. for 30 min, and diluted to 1:1024 with serum: DMEM=1:8 at a 2-fold ratio. Serum dilution+50 μL 100 TCID₅₀ PCV2 ZJ/c (TZ0601, NCBI serial number: EU257511.1) virus solution or PRV DX virus solution were neutralized at 37° C. for 2 h and then inoculated into a 96-well cell culture plate pre-populated with PK15 cells at 50 μL per well, incubated at 37° C. for 6 h and then transferred to 4% FBS DMEM medium. After 72 h, the cells were fixated to a cell plate and the number of positive wells was tested with PCV2 Cap monoclonal antibody IFA to measure the PCV2 neutralizing antibody titer; or after 72 h, the cell CPE was observed and the number of diseased wells was counted to measure the PRV neutralizing antibody titer.

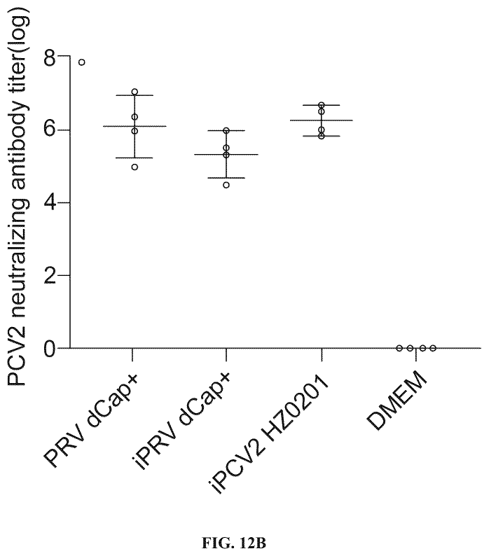

The results were shown in FIGS. 12A-B. Compared with the non-immune challenge control group, both the PRV dCap⁺ and iPRV dCap⁺ immunized groups produced higher PRV neutralizing antibody activity, where the neutralizing antibody activity produced by the PRV dCap⁺ group was better than that of the PRV HD/c control group. This indicated that the antibodies produced after the recombinant virus stimulating the animal body had desirable PRV neutralizing activity. Meanwhile, compared with the non-immune challenge control group, both the PRV dCap⁺ and iPRV dCap⁺ immunized groups also produced higher PCV2 neutralizing antibody activity. There was no statistical difference in neutralizing antibody activity between the two groups and the PCV2 inactivated vaccine (iPCV2 HZ0201) immunization control group. This indicated that the antibodies produced after immunizing animals with attenuated and inactivated recombinant viruses showed excellent neutralizing activity against PCV2.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A recombinant pseudorabies virus (PRV) particle expressing an exogenous immunogen on an envelope, wherein the recombinant PRV particle uses a gene corresponding to a non-essential envelope protein of a PRV as an insertion site, and replaces a gene sequence corresponding to an amino acid in an extramembrane domain of the non-essential envelope protein with a coding sequence corresponding to the exogenous immunogen.

2. The recombinant PRV particle according to claim 1, wherein the exogenous immunogen is derived from a pathogen.

3. The recombinant PRV particle according to claim 1, wherein the exogenous immunogen is derived from PCV2.

4. The recombinant PRV particle according to claim 2, wherein the exogenous immunogen is derived from PCV2.

5. The recombinant PRV particle according to claim 1, wherein the recombinant PRV particle uses a gE gene of the PRV as the insertion site; the gE gene comprises a gE initiation codon and a cytomegalovirus (CMV) promoter before the gE initiation codon; a modified enhanced green fluorescent protein (EGFP) gene is ligated after a gE termination codon of the gE gene, and two loxP sites in a same direction are inserted at both ends of the modified EGFP gene.

6. A transfer vector of the recombinant PRV particle according to claim 1.

7. A transfer vector of the recombinant PRV particle according to claim 2.

8. A transfer vector of the recombinant PRV particle according to claim 3.

9. A transfer vector of the recombinant PRV particle according to claim 4.

10. A transfer vector of the recombinant PRV particle according to claim 5.

11. A construction method of a recombinant PRV with green fluorescence, comprising the following steps: subjecting a eukaryotic cell to co-transfection using the transfer vector according to claim 6 and a genome of an attenuated PRV vaccine strain, and collecting a co-transfection mixture that has a lesion and EGFP green fluorescence; and
centrifuging the co-transfection mixture to collect a supernatant, and conducting plaque purification using the supernatant to obtain the recombinant PRV with green fluorescence.

12. A construction method of a recombinant PRV with green fluorescence, comprising the following steps: subjecting a eukaryotic cell to co-transfection using the transfer vector according to claim 7 and a genome of an attenuated PRV vaccine strain, and collecting a co-transfection mixture that has a lesion and EGFP green fluorescence; and
centrifuging the co-transfection mixture to collect a supernatant, and conducting plaque purification using the supernatant to obtain the recombinant PRV with green fluorescence.

13. A construction method of a recombinant PRV with green fluorescence, comprising the following steps: subjecting a eukaryotic cell to co-transfection using the transfer vector according to claim 8 and a genome of an attenuated PRV vaccine strain, and collecting a co-transfection mixture that has a lesion and EGFP green fluorescence; and
centrifuging the co-transfection mixture to collect a supernatant, and conducting plaque purification using the supernatant to obtain the recombinant PRV with green fluorescence.

14. A construction method of a recombinant PRV with green fluorescence, comprising the following steps: subjecting a eukaryotic cell to co-transfection using the transfer vector according to claim 9 and a genome of an attenuated PRV vaccine strain, and collecting a co-transfection mixture that has a lesion and EGFP green fluorescence; and
centrifuging the co-transfection mixture to collect a supernatant, and conducting plaque purification using the supernatant to obtain the recombinant PRV with green fluorescence.

15. A construction method of a recombinant PRV with green fluorescence, comprising the following steps: subjecting a eukaryotic cell to co-transfection using the transfer vector according to claim 10 and a genome of an attenuated PRV vaccine strain, and collecting a co-transfection mixture that has a lesion and EGFP green fluorescence; and
centrifuging the co-transfection mixture to collect a supernatant, and conducting plaque purification using the supernatant to obtain the recombinant PRV with green fluorescence.

16. A recombinant PRV with green fluorescence constructed by the construction method according to claim 11.

17. A recombinant PRV with green fluorescence constructed by the construction method according to claim 12.

18. A construction method of a recombinant PRV without fluorescence, comprising the following steps: extracting a genome of the recombinant PRV with green fluorescence according to claim 16, and mixing the genome with a Cre enzyme to allow enzymatic digestion in vitro; transfecting a resulting enzymatic digestion product into a eukaryotic cell, and collecting a co-transfection mixture that has a lesion but no fluorescence; selecting a clone with no fluorescence but showing the lesion by conducting plaque screening on the co-transfection mixture to obtain the recombinant PRV without fluorescence.

19. A recombinant PRV without fluorescence constructed by the construction method according to claim 18.

20. A preparation method of a PRV/circovirus (CV)-based bivalent vaccine or polyvalent vaccine using the transfer vector according to claim 6.
